(19) United States
(12) Patent Application Publication
(10) Pub. No.: US 2007/0156138 A1
(43) Pub. Date: Jul. 5, 2007

(54) Apparatus for the interstitial coagulation of tissue

(75) Inventor: Florian Eisele, Tubingen (DE)

Correspondence Address: AKIN GUMP STRAUSS HAUER & FELD L.L.P., ONE COMMERCE SQUARE, 2005 MARKET STREET, SUITE 2200, PHILADELPHIA, PA, 19103, US

(73) Assignee: ERBE ELEKTROMEDIZIN GMBH, Tubingen (DE)

(21) Appl. No.: 10/598,229

(22) PCT Filed: Feb. 25, 2005
(86) PCT No.: PCT/EP05/02001

§ 371 Date: Aug. 22, 2006

(30) Foreign Application Priority Data
Feb. 25, 2004 (DE) 10 2004 009 206.0
Mar. 16, 2004 (DE) 10 2004 012 813.8

(51) Int. Cl. A61B 18/14

(52) U.S. Cl. 606/49; 606/45

(57) ABSTRACT

An apparatus for the interstitial coagulation of tissues includes at least one electrode by way of which an HF coagulation current can be conducted into the tissue of a patient. The electrode is constructed as a three-dimensional body that can be expanded to various states of expansion, or is attached to such a body, so that by continuous or stepwise expansion of the body the electrode can be kept in constant electrical contact with the tissue during coagulation. In this way the apparatus enables the tissue treatment to be more uniformly distributed.

### CROSS-REFERENCE TO RELATED APPLICATIONS

This application is a section 371 of International Application No. PCT/EP2005/002001, filed Feb. 25, 2005, which was published in the German language on Sep. 1, 2005, under International Publication No. WO 2005/079688 A1 and the disclosure of which is incorporated herein by reference.

### BACKGROUND OF THE INVENTION

The invention relates to an apparatus for the interstitial coagulation of tissue.

In high-frequency surgery, in particular in the area of endoscopic applications, various devices are known by means of which the electrical energy can be sent from a high-frequency surgical appliance, i.e. a HF unit, to the particular tissue sites that are to be treated.

Electrodes are known that apply the energy directly, by making contact with the tissue. These present the problem that in some circumstances the tissue becomes heated to such an extent that it is carbonized, i.e. is completely destroyed by burning.

Another problem with such electrodes resides in the fact that the electrodes can adhere to the tissue. If this occurs, during removal of the electrode the tissue will be torn apart. To reduce the risk of adhesion, the electrodes are often coated with an appropriate substance. Such electrodes are known, e.g., from the document DE 199 41 105 C2.

The problems described above arise in particular where interstitial coagulation is involved, for instance the coagulation of a hepatic tumor, because in such cases the electrode is inserted into the tissue to be treated, e.g. by piercing it, and as a result is completely surrounded by tissue. The danger that the tissue will be burnt and/or will adhere to the electrode is present here to a particularly high degree. Should that occur, because of the large resistance at the contact between electrode and tissue an evenly distributed devitalisation may no longer be possible; that is, the tissue is not uniformly coagulated.

Another problematic aspect is that during the interstitial coagulation the tissue contracts (dries out), so that the electrode is no longer apposed to it. A gap that appears between the electrode and the tissue to be treated, as a result of the tumor desiccation, increases the contact resistance between electrode and tissue so that the extent to which electrical energy can be introduced is limited. This, too, results in nonuniform coagulation or can even prevent the occurrence of any coagulation effect.

From the document U.S. Pat. No. 6,090,105 A, for example, an ablation apparatus is known that comprises an introducer electrode to be inserted into a tissue that is to be treated. In the interior of the introducer electrode are disposed additional electrodes, which can be pushed out of the introducer electrode once the insertion has been completed, and thus can positioned within the tissue. The electrodes are made of a memory alloy and are placed under tension at temperatures above a certain level. When outside the introducer electrode, these electrodes tend to expand to their original shape and hence penetrate into the surrounding tissue during a coagulation procedure. With the appliance just described, the problems discussed above are even more likely to occur. Furthermore, electrode arrangements of this kind make it particularly difficult to achieve a uniform coagulation volume, because the electrodes penetrate into the tissue in an uncoordinated manner, depending only on the intrinsic force that tends to expand them.

### BRIEF SUMMARY OF THE INVENTION

Embodiments of the present invention provide an apparatus for the interstitial coagulation of tissue such that tissue can be treated with greater uniformity.

According to the invention there is provided an apparatus for the interstitial coagulation of tissues comprising a three-dimensional body that can be expanded to various states of expansion, and at least one electrode adapted to conduct an HF coagulation current into the tissue, the electrode forming at least a part of the three-dimensional body such that by one of continuous and stepwise expansion of the body the electrode can be kept in constant electrical contact with the tissue during coagulation. The electrode is itself constructed either as the three-dimensional body that can be expanded to different degrees, or is attached to such a body.

The invention enables the three-dimensional body, i.e. the electrode itself or an expandable body on which the electrode is disposed, to be inserted into the tissue to be treated while in a non-expanded state, and during coagulation to be expanded in a controlled manner according to the degree of coagulation, i.e. in dependence on defined conditions. Hence the body is constructed and can be actuated in such a way that during coagulation it follows, in a controlled manner, the treated tissue, which as a rule is moving away from the electrodes. This expansion of the body can occur automatically or be manually induced, and is carried out on the basis of detected parameters that represent a measure for the degree of coagulation. The controlled coagulation enables a uniform tissue devitalisation. As the electrode continually expands, furthermore, it exerts a pulling force that smooths out the treated tissue during the coagulation process, so that this aspect itself ensures a uniform coagulation.

The apparatus for interstitial coagulation can, for example, be inserted into the tissue to be treated by means of an attachment suitable for piercing the tissue, or alternatively the device itself is constructed so that it can pierce the tissue. The device can be designed either for direct application or for endoscopic use.

In a first preferred embodiment, a control device is provided to control the amount of expansion of the body in dependence on the coagulation current. The control device is preferably incorporated into a HF-surgery appliance or, alternatively, provided as an external component. The control device is furthermore incorporated into the apparatus, i.e. in principle into the electrosurgical instrument, and is designed to detect the diverse parameters that reflect the degree of coagulation, in this case the coagulation current. Therefore it is possible, for example, to determine that the contact resistance has increased owing to the above-mentioned formation of a gap between electrode and tissue, by detecting the resulting decrease in strength of the coagulation current. The control device is also designed so that the body can then be controlled on the basis of the detected parameter, i.e. on the basis of the detected current strength, in such a way that the degree of expansion of the body changes in dependence on the detected parameter. Hence in this case the body is expanded until the gap has been closed, so that electrode and tissue can be kept in the electrical contact necessary for the subsequent coagulation procedure. Therefore an optimal coagulation result is ensured in every phase of coagulation.

Alternatively, it is possible to construct the control device in such a way that information about the detected value, i.e. in this case the detected current intensity, is made available by way, e.g., of a visual display. An operator can then control the expansion of the body manually on the basis of this information.

The control device is preferably provided with a transducer that measures the coagulation current and transmits the measured values to the control device. Reliable assessment of the current can also be achieved, e.g., with a current monitor such as is known in the state of the art.

That a gap is beginning to form can, alternatively, be detected by a pressure measurement. For this purpose the control device, where appropriate incorporating the measured-value transducer, is designed so that, e.g., the pressure exerted by the target tissue against the electrode is measured, and when this pressure falls below a specified threshold, the body should be controlled so that it expands sufficiently to preserve a suitable distance between electrode and tissue. The measured-value transducer in this case is preferably an electronic pressure sensor. It is also possible to employ a Hall-effect sensor, which detects the change in a magnetic field as a function of the electrode excursion.

When the coagulation current is measured simultaneously with the pressure acting on the electrode, it is furthermore possible to obtain a prediction as to whether the tissue will be burnt to an undesired extent, or whether adhesion of the electrode will occur. That is, if a suitable pressure is measured but the current is simultaneously decreasing, it should be concluded that the decrease in current intensity is not due to formation of a gap between electrode and tissue.

In a second preferred embodiment the control device is designed and disposed in such a way that the coagulation current between electrode and tissue can be set to a particular current density. In each state of expansion of the three-dimensional body, it forms a specific electrode surface area. Given a constant current intensity, a current density decreases progressively as the body expands, because the electrode area becomes larger. Therefore the control device can be used to adjust the current density to the value required for an optimal coagulation result, and the current intensity can be subsequently adjusted, so that even when the electrode surface is increasing, an optimal coagulation result is ensured at every time during the coagulation process. This adjustment of the current intensity can be accomplished automatically or also manually.

The measured current density and, when required, a subsequent regulation of the coagulation current provide a measure of the state of expansion of the body and hence also a measure of the degree of coagulation. When the current density falls below a prespecified threshold value, the coagulation of a defined target tissue region has been completed. For coagulation to be continued, the body would have to be expanded, and then the renewed coagulation would again be observable by a change in the current density. Then adjustment to an appropriate current intensity can be carried out manually. It is also possible, however, to design the control device in such a way that the current intensity is set automatically.

The control device is preferably designed so that the current density can be adjusted independently, regardless of the state of expansion of the body. Hence different coagulation strengths are available for different kinds of tissue or stages of coagulation. For example, if unexpected bleeding occurs, an increase in the current density can produce stronger coagulation.

One solution in accordance with the invention is to provide measurement devices with which to determine the state of expansion of the body. Thus conclusions can be drawn about the degree of coagulation at all times, if the expansion is being automatically controlled. For example, the state of expansion can be output by way of a visual display. When the body is being manually expanded, the operator can make subsequent adjustments in accordance with this display. As measurement devices here, the above-mentioned measured-value transducers can be employed.

Preferably the electrode component of the apparatus comprises a treatment electrode that is at least to some extent permeable to liquids and can be brought into contact with a section of the tissue, as well as a liquid-supply device through which an electrically conductive liquid can be delivered to the treatment electrode, and a current-supply device to deliver the HF coagulation current to the treatment electrode in such a way that the HF treatment current can be applied to the liquid passing through the treatment electrode. An instrument thus constructed therefore preferably comprises an expandable hollow body that while in a non-expanded state can be inserted into the tissue to be treated, and during the coagulation process can follow the shrinking, coagulated tissue as a result of its own expansion. At the same time, in addition to the fact that the tissue is thereby smoothed out, this design offers the advantage that adhesion of the electrode to the tissue is effectively prevented because the current is conducted by means of the liquid. The electrical conductivity of the liquid, i.e. solution (e.g. Ringer solution or pure table-salt solution), causes the HF current to reach the tissue site that is to be treated. The solution simultaneously cools the tissue during the treatment, so that it becomes hardly any warmer than the boiling point of the solution.

In one preferred embodiment the treatment electrode comprises an elastically extensible or foldable surface element on the inside of which, i.e. the side opposite to the tissue, there is disposed an interior space within which internal pressure can be applied, so that the surface element can be expanded by increasing the internal pressure. The surface element makes possible a uniformly distributed contact between the electrode and the tissue to be treated, and facilitates maintenance of the electrical contact between electrode and tissue so as to achieve a uniform devitalisation. The desired expansion of the body, in this case the surface element, during coagulation is produced in the simplest manner, by increasing the pressure within the interior space.

Preferably the surface element has the shape of a ring or sphere. Then the treatment electrode can be constructed, e.g., in the form of a balloon catheter, i.e. a form already familiar to the operator.

In one embodiment of the invention the interior space is filled with the electrically conductive liquid. In this arrangement, therefore, the electrically conductive liquid simultaneously serves as a medium with which to generate the internal pressure that stretches/inflates the surface element. In this case the treatment electrode is constructed so that its resistance to flow of the electrically conductive liquid is large enough that on one hand an internal pressure can be built up, while on the other hand the amount of liquid that emerges is sufficient to provide a reliable electrical contact. For this purpose the treatment electrode can incorporate a piece of film, felt or woven fabric that permits the required amount of liquid flow. In any case it is advantageous for the treatment electrode to consist substantially of a thermally stable material, in particular a tetrafluoroethylene material.

Preferably the electrically conductive fluid includes polyvinyl pyrrolidone (PVP), a surfactant or a similar substance to alter the viscosity of the electrically conductive fluid without any side effects that would be deleterious to the intended treatment.

The employment of a fluid that diffuses through the expandable electrode does not exclude the possibility that the electrode surface itself can also be electrically conductive. One possible embodiment is an electrode made of an elastomer that has been made conductive, for instance, by the incorporation of metal particles.

The internal pressure can be made independent of the pressure with which the liquid is pushed through the treatment electrode by an arrangement such that the interior space contains an expandable accessory body that is hydraulically separated from the electrically conductive liquid. As a result, it can be ensured that even during extreme expansion not too much electrically conductive liquid emerges. In particular, the surface element can be constructed in several layers, so that in an inner layer liquid flows in the surface direction with a reduced resistance to flow, while in an outer layer it can be conducted perpendicular to the surface direction. Such an arrangement can be relatively simply produced. To increase the uniformity of the liquid output, a separation layer with larger resistance to flow is positioned between the inner and the outer layer.

In all cases the current is introduced into the electrically conductive liquid by means of a conductor with low electrical resistance, e.g. a wire, at a position as close as possible to the treatment electrode.

In order to avoid problems caused by the inevitable excess of electrically conductive liquid that emerges, in one preferred embodiment of the invention a suction device is provided to suck away excess liquid.

The electrosurgical instrument, i.e. the apparatus, can be constructed as a monopolar coagulation instrument, with only a single current-supply lead, while the tissue to be treated (i.e., the patient) is kept at the other potential. In another preferred embodiment of the invention a bipolar application can be employed, by using two electrodes that are in principle identical. That is, the treatment electrode is constructed with at least two sections that are electrically insulated from one another, thus forming a bipolar electrode, so that no neutral electrode needs to be attached to the patient.

Preferably the electrode is constructed so that a cutting current can be applied to it. This reduces the risk that cancer cells will be released when the electrode is inserted into a tumor.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 is a functional block diagram showing a HF-surgery arrangement with an apparatus for interstial coagulation;

FIG. 2 shows a first embodiment of the apparatus in a partially sectional, perspective representation;

FIG. 3 shows a second embodiment of the apparatus in longitudinal section;

FIG. 4 shows a third embodiment of the apparatus in longitudinal section and

FIG. 5 shows an enlarged sectional representation of the region V in FIG. 4.

### DETAILED DESCRIPTION OF THE INVENTION

In the following description, the same reference numerals are used for identical parts or parts with identical actions.

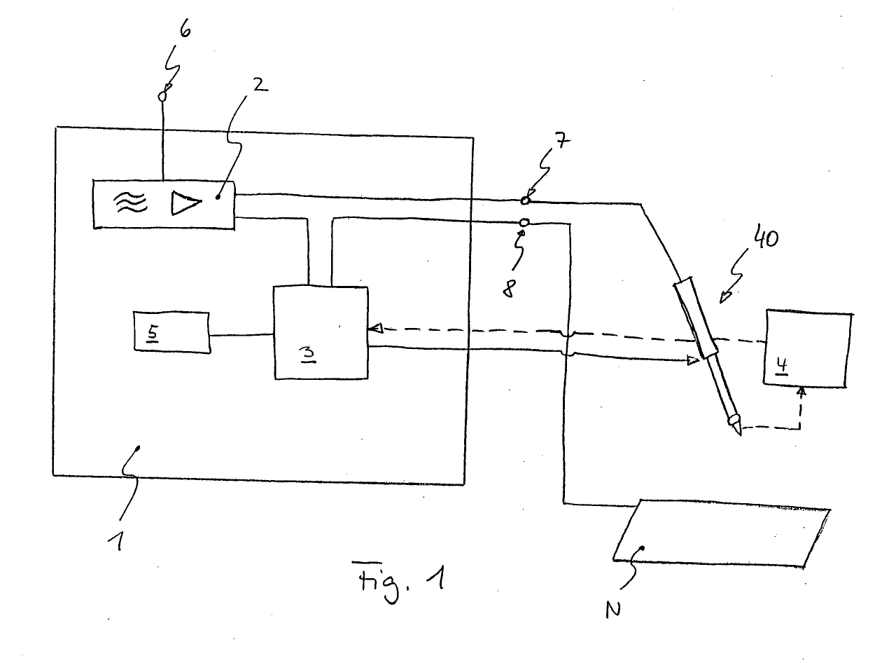

The functional block diagram in FIG. 1 represents a HF-surgery arrangement with an apparatus for interstitial coagulation. Here the components of such a HF-surgery arrangement that are important for explaining the invention are shown schematically, including the apparatus 40 for interstitial coagulation.

In monopolar arrangements a HF current produced by a HF generator 2 is sent to an electrosurgical instrument 40 (in this case the apparatus for interstitial coagulation), which by way of an active electrode 10, i.e. a monopolar coagulation electrode, applies the current to a tissue that is to be treated; in this process the current passes through the body of a patient to the neutral electrode N and from there back to the HF generator 2. Bipolar arrangements do not need any neutral electrode, because the current path runs between two electrodes of an electrosurgical instrument.

A HF-surgery appliance 1 comprises an input connector 6 to which finger- and/or foot-operated switching devices (not shown) are connected. These switching devices enable the HF current to be activated and/or inactivated. The switching devices here are preferably embodied by a computer arrangement. On the output side the HF-surgery appliance 1 is provided with a first output connector 7 and a second output connector 8, by way of which the monopolar coagulation instrument 40 can be connected to the associated neutral electrode N. It is also possible for a bipolar electrosurgical instrument (not shown) to be connected to the HF-surgery appliance 1. For practical purposes, a HF-surgery appliance is usually designed with different connectors, for mono- or bipolar electrodes. The neutral electrode N is shown schematically and in practice completely covers a section of the patient's body.

At the core of the HF-surgery appliance 1 is the controllable HF generator 2, to produce a HF voltage and conduct the HF current to the coagulation electrode 10 of the electrosurgical instrument 40. The HF generator 2 is connected to a control device 3 for controlling the electrode 10 of the electrosurgical instrument 40. A measured-value transducer 4 associated with the control device 3 is likewise shown here, and is provided to detect various parameters that serve as a measure of the degree of coagulation, e.g. a contact resistance between the electrode 10 and the tissue.

The electrosurgical instrument 40, i.e. the apparatus, is constructed with at least one electrode 10 through which the HF coagulation current can be conducted into the tissue. The electrode 10 is designed as a three-dimensional body that can be expanded to various expansion states, or is attached to such a body, so that by continuous or stepwise expansion of the body the electrode 10 can be kept in constant electrical contact with the tissue during the coagulation.

The expandable electrode body 10, or an expandable body 14 to which the electrode 10 is attached, is constructed and can be actuated in such a way that it is inserted into the tissue to be treated while in the non-expanded state, and during the (in particular interstitial) coagulation process it is expanded so that as the tissue shrinks away from the electrode, as is usually the case, the electrode follows it in a controlled manner. In this process the expansion of the body can be brought about automatically or manually and may depend on detected parameters that represent a measure of the degree of coagulation. The controlled coagulation enables a uniform tissue devitalisation.

The apparatus for interstitial coagulation with the expandable body 10, 14 can, for example, be inserted into the tissue by means of an insertion device (not shown) suitable for piercing the tissue; alternatively, however, the apparatus 40 can itself be constructed so that it is able to pierce the tissue. The apparatus 40 can be designed for direct employment or for endoscopic application.

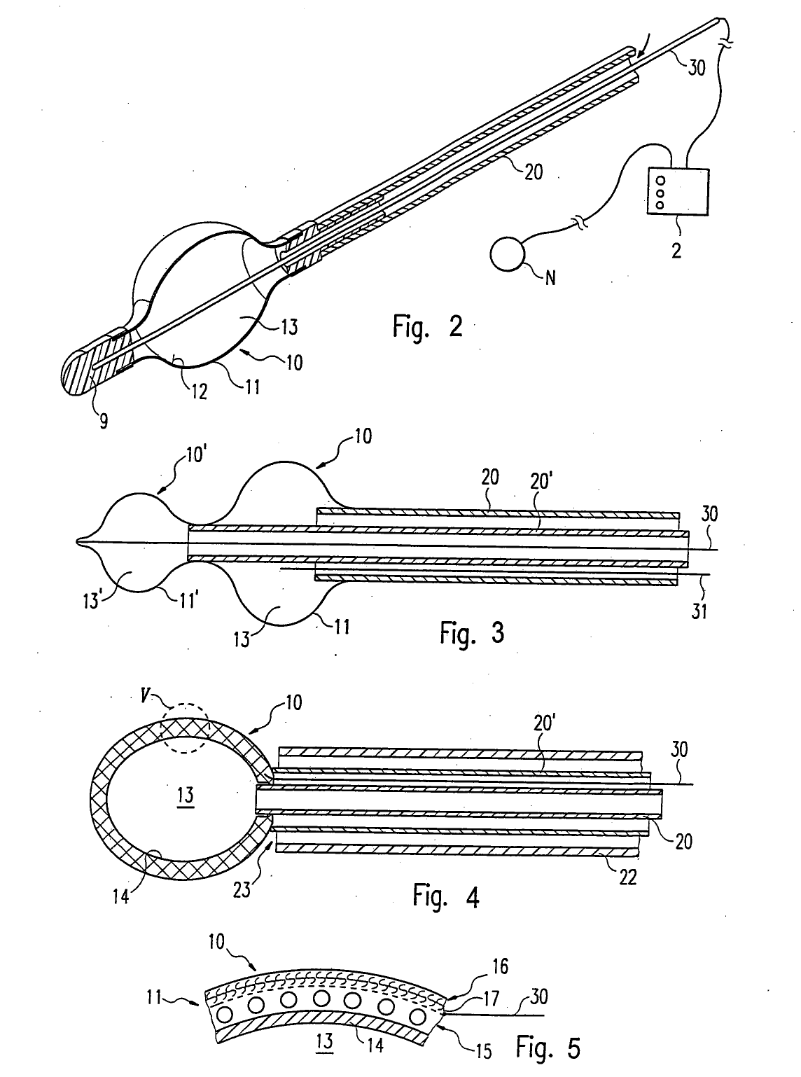

Special embodiments of the electrosurgical instruments 40 and the associated electrodes are explained in greater detail with reference to FIGS. 2 to 5.

During interstitial coagulation the tissue dries out and therefore contracts owing to the coagulation. Accordingly, the electrode 10 is no longer in contact with the tissue to be treated, and the formation of this gap causes a contact resistance between electrode 10 and tissue to increase. The result is that the intensity of the coagulation current decreases.

The control device 3 here is designed so that it detects this reduction in current intensity and then controls the electrosurgical instrument 40 in such a way that the body 10, 14 expands so as to follow the tissue. In this case, therefore, the body 10, 14 is expanded until the gap has been closed, so that the electrical contact between electrode 10 and tissue can be maintained as is necessary for the coagulation process to continue. Hence in every phase of coagulation an optimal coagulation result is guaranteed. The detected current values can be output, e.g., by means of a display 5, so that an operator can visually monitor the decrease in current strength. With this information available, the expansion of the body 10, 14 can also be performed under manual control.

Alternatively, it is possible for the measured-value transducer 4 associated with the control device 3 to detect other parameters that indicate the degree of coagulation, for example the contact resistance itself.

An onset of gap formation can alternatively be detected by means of a pressure measurement. For this purpose the control device 3 is designed so that, e.g., the pressure exerted by the target tissue against the electrode 10 is measured, and when this pressure falls below a prespecified threshold the body 10, 14 should be controlled so that it expands sufficiently to preserve a suitable distance between electrode 10 and tissue. In this case the measured-value transducer 4 is preferably constructed as an electronic pressure sensor. It is also possible to employ a Hall-effect sensor, which detects the change in a magnetic field in dependence on the electrode excursion.

The control device 3 can also be disposed and designed in such a way that the coagulation current between electrode 10 and tissue can be adjusted to obtain a particular current density. The three-dimensional body 10, 14 in each state of expansion describes a specific electrode surface. If the current strength is kept constant, a current density decreases as the body 10, 14 progressively expands, because the electrode surface is increasing. Therefore the control device can be used to set a current density that is required for an optimal coagulation result and to alter the current intensity accordingly, so that even when the electrode surface is becoming larger, an optimal coagulation result is guaranteed at each time during the coagulation process.

Here, again, the adjustment of current strength can be performed both automatically and manually. Because the current density ultimately reflects the state of expansion of the body 10, 14, the surgeon can, for example, induce expansion of the body 10, 14 for declining current density.

In order to allow for differences in the kinds of tissue, stages of coagulation and/or unpredictable events such as severe bleeding, the control device 3 is designed so that the current intensity and/or current density can be adjusted independently of the state of expansion. For instance, if stronger coagulation is needed this can be achieved by increasing the current density, regardless of the expansion state.

If measurement devices are provided to detect the state of expansion of the body 10, 14, this information can be made available, e.g., by way of the display 5, preferably a visual display. If expansion of the body 10, 14 is to be induced manually, the operator can make the appropriate adjustments by referring to the display 5. The measurement devices can be embodied by the measured-value transducer described above, or be provided as an independent instrument.

FIGS. 2 to 5 show various kinds of apparatus 40 for interstitial coagulation. In the drawings these are designed for insertion into a working channel of an endoscope. The endoscope must then be constructed so as to permit piercing of the tissue to be treated. However, the instruments can also be designed so that they themselves are suitable for piercing.

As shown in FIG. 2, the apparatus comprises the treatment electrode 10, shown here in an expanded state, which as a whole is constructed substantially as a stretchable surface element 11. By way of a flexible or rigid supply tube 20 an electrically conductive liquid (indicated by an arrow), e.g. Ringer solution or a solution of table salt, can be passed through the supply tube 20 into an interior space 13 of the treatment electrode 10. The stretchable or unfoldable surface element 11 here is sufficiently porous that liquid entering the interior space 13 can pass from an inside 12 of the stretchable surface element 11 through the treatment electrode 10, to the outside.

In the interior space 13 there is a supply electrode 30 in the form of a wire, which at a distal end of the treatment electrode 10 is mechanically connected to the treatment electrode 10 by way of an electrically insulating end piece 9. The supply electrode 30 is connected to the HF generator (not shown here).

In order to perform an interstitial coagulation, for instance to insert the electrode into a tumor, the entire apparatus is pushed for example through the working channel of an endoscope that had previously been positioned at the site to be treated; while being thus installed, the surface element is still in a collapsed state rather than being inflated as shown in FIG. 2. An instrument tip can be configured in such a way that it can be moved forward like a thorn, forcing the tissue aside. That is advantageous when, on the way to the target tissue, other tissue structures must be passed while causing the least possible damage. For positioning in the target tissue it can be advantageous to apply a HF cutting current to the electrode. This can reduce the danger that cancer cells will be set free when the electrode is inserted into a tumor. Furthermore the electrode, because it is free from mechanical tension, can be precisely positioned. As soon as the electrode is in position, electrically conductive liquid (e.g., Ringer solution) is passed through the supply tube 20 in the direction indicated by the arrow in FIG. 2, in which process the pressure is adjusted so that the treatment electrode 10, i.e. its stretchable surface element 11, can be expanded. Now in order to coagulate the tumor from its interior outward, the HF generator 2 is actuated so that a coagulation current flows through the liquid into the tissue to be treated and devitalizes that tissue. Because electrically conductive liquid is continuously being supplied and emerging through the stretchable surface element 11, it is ensured that a film (or cushion) of liquid will constantly be present between the stretchable surface element 11 and the tissue to be treated; on one hand this liquid serves to conduct the HF current while on the other hand it cools the surface of the tissue and protects it from adhering to the treatment electrode 10. The surface element 11 is progressively expanded during the course of the coagulation and thus—as described above—follows the retreating tissue.

In the embodiment of the invention described above, the HF current is supplied in a monopolar arrangement. Therefore a neutral electrode N is applied to the patient to serve as the opposite pole. The embodiment of the invention shown in FIG. 3 differs from that in FIG. 2 in that the instrument as a whole has a bipolar construction. For this purpose there are provided two treatment electrodes 10, 10′ with corresponding stretchable surface elements 11, 11′, in an arrangement analogous to that in FIG. 2 (coaxial). In accordance with the presence of two treatment electrodes 10, 10′ and stretchable surface elements 11, 11′, there are two supply tubes 20, 20′ and two supply electrodes 30, 31, so that the interior spaces 13, 13′ can be placed under pressure independently of one another, and the stretchable surface elements 11, 11′ can be expanded to different degrees.

The embodiment shown in FIG. 4 differs from those previously described in that the interior space 13 of the treatment electrode 10 is enclosed by an elastic, stretchable auxiliary body 14, which is completely water-tight. The stretchable surface element 11 consists—as shown in FIG. 5—of several layers, including an inner layer 15 through which, having been supplied by way of a conduit 20′, the electrically conductive fluid moves in the direction of the surface, and which in this direction has a relatively low resistance to flow. On its outer side the treatment electrode 10 bears an outer layer 16 which conducts the liquid primarily in a direction perpendicular to the surface. Between the inner layer 15 and the outer layer 16 is a partition layer 17, which presents a greater resistance to flow than does the inner layer 15 but nevertheless allows liquid to pass from the inner layer 15 into the outer layer 16. In the embodiment of the invention shown here this partition layer 17 is simultaneously an electrically conductive layer that is in electrical contact with the supply electrode 30. In this arrangement, therefore, the treatment electrode 10 and hence the stretchable surface element 11 can be expanded by the auxiliary body 14, and in this case not only liquid but also gas can be used to produce the expansion. Regardless of the state of expansion, then, the electrically conductive liquid can be supplied and uniformly distributed over the inner layer 15. The amount of liquid that emerges is then determined exclusively by the pressure with which the electrically conductive liquid is supplied, and hence is independent of the state of expansion of the treatment electrode 10.

In this embodiment there is additionally provided a suction tube 22 with a suction opening 23 in the vicinity of the treatment electrode 10. By way of this suction tube 22 the excess electrically conducting liquid can be sucked away.

From the above it will be evident that the described characteristics can also be combined in the various embodiments. For example, a suction tube can be provided in all embodiments of the invention. Similarly, it is also possible to employ an auxiliary body 14 in the case of the embodiment shown in FIG. 2.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. An apparatus for the interstitial coagulation of tissue, comprising a three-dimensional body that can be expanded to various states of expansion, and at least one electrode adapted to conduct an HF coagulation current into said tissue, the electrode forming at least a part of said three-dimensional body such that by one of continuous and stepwise expansion of said body said electrode can be kept in constant electrical contact with the tissue during coagulation.

2. The apparatus according to claim 1, wherein a control device is provided for controlling the degree of expansion of the body dependent on said coagulation current.

3. The apparatus according to claim 2, wherein said control device is adapted to enable an adjustment of a current density of said coagulation current between said electrode and said tissue.

4. The apparatus according to claim 3, wherein said control device permits the current density to be adjusted independently of the degree of expansion.

5. The apparatus according to claim 1 wherein measurement devices are provided for detecting the state of expansion of said three-dimensional body.

6. The apparatus according to claim 1, wherein said electrode comprises a treatment electrode that is at least partially permeable to liquid and that can be brought into contact with a section of said tissue, and comprising a liquid-supply device through which an electrically conductive liquid can be delivered to said treatment electrode, and a current supply device adapted to deliver said HF coagulation current to said treatment electrode in such a way that said HF treatment current is conducted to the liquid that is passing through the treatment electrode.

7. The apparatus according to claim 6, wherein said treatment electrode comprises one of an elastically stretchable and an unfoldable surface element that defines an interior space to which an internal pressure can be applied to expand said surface element by increasing the internal pressure.

8. The apparatus according to claim 7, wherein said surface element is in the form of one of a ring and a sphere.

9. The apparatus according to claim 6, wherein said treatment electrode is constructed in the form of a balloon catheter.

10. The apparatus according to claim 7, wherein said interior space is adapted to be filled with said electrically conductive liquid.

11. The apparatus according to claim 6, wherein said electrically conductive liquid comprises one of polyvinyl pyrrolidone (PVP), a surfactant and a similar means of changing the viscosity of said electrically conductive liquid.

12. The apparatus according to claim 6, wherein said treatment electrode is made of a thermally stable material in the form of one of a film, a felt and a woven fabric.

13. The apparatus according to claim 7, wherein said interior space is enclosed by an expandable auxiliary body that is hydraulically separated from said electrically conductive liquid, and said surface element is constructed in several layers such that in an inner layer liquid can be conducted in a direction towards an outer surface of the element and in an outer layer liquid can be conducted in a direction perpendicular to the surface direction.

14. The apparatus according to claim 6, wherein a suction device is provided to suck away liquid.

15. The apparatus according to claim 1, wherein said electrode is adapted to be supplied with a cutting current.

16. The apparatus as claimed in claim 12, wherein said thermally stable material is comprised of tetrafluoroethylene.

17. The apparatus as claimed in claim 13, wherein a partition layer with a greater resistance to liquid flow than said inner layer is disposed between said inner layer and said outer layer.
